(19) United States
(12) Patent Application Publication
(10) Pub. No.: US 2012/0041312 A1
(43) Pub. Date: Feb. 16, 2012

(54) Method for Improving Image Quality of Ultrasonic Image, Ultrasonic Diagnosis Device, and Program for Improving Image Quality

(75) Inventors: Kenji Nakahira, Fujisawa (JP); Atsushi Miyamoto, Yokohama (JP)

(73) Assignee: Hitachi Medical Corporation, Chiyoda-ku, Tokyo (JP)

(21) Appl. No.: 13/145,129

(22) PCT Filed: Apr. 26, 2010
(86) PCT No.: PCT/JP2010/002976

§ 371 Date: Jul. 19, 2011

(30) Foreign Application Priority Data
Apr. 28, 2009 (JP) 2009-108905

(51) Int. Cl. A61B 8/14; G06K 9/00

(52) U.S. Cl. 600/443; 382/131

(57) ABSTRACT

In an ultrasonic diagnosis device, while a signal component is preserved, a factor of degrading image quality such as a flicker of a noise is suppressed. An input image is separated into a signal component image and a noise component image. After frame synthesis processing is performed on the noise component image, the signal component image is synthesized with the noise component image having undergone the frame synthesis. Thus, the noise is suppressed. Otherwise, after the input image is separated into the signal component image and noise component image, the frame synthesis processing is performed on the signal component image. The noise component image is then synthesized with the signal component image having undergone the frame synthesis. Thus, discernment of a signal can be improved.

### TECHNICAL FIELD

The present invention relates to an ultrasonic diagnosis device that acquires an image by transmitting or receiving ultrasonic waves to or from a subject. More particularly, the invention is concerned with a technology for performing image quality improvement processing, which is image processing, on an acquired image.

### BACKGROUND ART

In an ultrasonic image picked up by an ultrasonic diagnosis device, a noise generally called a speckle noise is mixed. The speckle noise is thought to occur in an entire image due to interference of plural reflected waves returned from microscopic structures in a living body. Under certain conditions for imaging, an electric noise or thermal noise of a non-negligible level may be mixed in the ultrasonic image. The noises bring about degradation in image quality such as a flicker in an image, and become a factor of disturbing a signal component that should be observed.

As for types of ultrasonic images, there are cited a B (brightness)-mode image produced by converting reflectance levels of a living tissue of a subject into lightness levels of pixel values, a Doppler image that provides moving-speed information concerning the living tissue, a color flow mapping image produced by coloring part of the B-mode image that expresses a motion, a tissular elasticity image that provides hue information dependent on a magnitude of distortion of the living tissue or an elastic modulus thereof, and a synthetic image produced by synthesizing pieces of information on these images. Minimization of noises in the images is desired.

The shape of a speckle noise irregularly varies depending on the position of a microscopic structure in a living body. As already known, even when a tissue makes a little motion, the pattern of the speckle noise largely varies. In addition, the pattern of an electric noise or thermal noise changes imaging by imaging. In existing ultrasonic diagnosis devices, plural frames of a pickup image are used to perform frame synthesis processing in order to reduce a noise component that expresses an impetuous motion.

When frame synthesis processing is applied more intensely, a noise component can be more effectively reduced. However, a problem that a signal component deteriorates becomes outstanding. In efforts to cope with the problem, a proposal has been made of a system in which frame synthesis processing is not performed using a fixed weight, but the weight is calculated based on a degree of a change in a brightness value, and used to perform the frame synthesis processing (refer to, for example, patent literatures 1 to 3).

In addition, the frame synthesis processing has proved effective in suppressing a flicker caused by a signal component of a relatively high moving speed, and suppressing a quasi-pattern that stems from other image processing such as edge enhancement processing.

### CITATION LIST

### Patent literature

Patent literature 1: Japanese Patent Application Laid-Open Publication No. 8-322835

Patent literature 2: Japanese Patent Application Laid-Open Publication No. 2002-301078

Patent literature 3: Japanese Patent Application Laid-Open Publication No. 2005-288021

### SUMMARY OF INVENTION

### Technical Problem

However, according to the conventional systems, reduction of a noise component and explicit display of a signal component cannot be, as cited below, fully realized.

(1) In order to intensely suppress a noise component with a signal component preserved, it is necessary to highly precisely discriminate the signal component from the noise component on the basis of some index so that frame synthesis processing can be feebly applied to the signal component and intensely applied to the noise component. However, according to the conventional system of performing the discrimination using a degree of a change in a brightness value, it is hard to highly precisely discriminate the signal component from the noise component.

(2) Due to a factor such as relative positions of an ultrasonic probe and a signal component, the signal component is not always explicitly rendered in each frame. A border of a tissue may be seen discontinuously or the frame is seen as if part of the signal component were lost. In order to improve the discernment of the signal component, frame synthesis processing is preferably performed on the signal component to some extent. However, for a signal component that expresses a motion, since a positional deviation occurs between frames, when frame synthesis alone is performed, bluntness of the signal component ensues.

(3) In order to improve discernment without blunting a signal component, different processing is preferably performed according to the speed of a motion of the signal component. In some cases, it may be preferred that different processing is performed between a microscopic signal component whose bluntness is likely to be noticed and other signal components. Likewise, as for a noise component, different processing is preferably performed between a component expressing an impetuous motion and a component expressing a moderate motion or between a speckle noise and other noises. According to the conventional system, it is hard to perform appropriate processing according to the type of signal component or noise component.

(4) An optimal frame synthesis processing method varies depending on a scan rate or a region to be imaged. For example, when the scan rate is high, frame synthesis processing should be performed using a larger number of frames. This is because a noise can be intensely suppressed with a signal preserved and a high-quality image can be obtained. According to a method in which the intensity of frame synthesis processing is fixed but does not depend on the scan rate or region to be imaged, it is hard to output a high-quality image under various conditions all the time.

(5) In order to ensure image quality that is fully satisfactory to a purpose of observation or to a user, it is necessary to appropriately designate parameters for image quality improvement processing. However, it is hard for the user to understand the meanings of many parameters. It is hard to appropriately designate the parameters.

An object of the present invention is to provide a method for improving image quality which makes it possible to provide a satisfactory effect of image quality improvement even in the foregoing case, an ultrasonic diagnosis device, and a processing program thereof.

### Solution to Problem

In the present invention, the object is accomplished by a method for improving image quality in an ultrasonic diagnosis device, an ultrasonic diagnosis device in which the method for improving image quality is implemented, and a processing program thereof.

The present invention is a method for improving image quality of a pickup image of an ultrasonic diagnosis device, and is characterized in that: the pickup image is separated into two or more images; on at least one of the separate images, frame synthesis processing is performed together with corresponding separate images in one or more frames of an ultrasonic image of different time phases; and an image obtained through the frame synthesis processing is synthesized with the separate image.

Further, the present invention is a method for improving image quality of a pickup image of an ultrasonic diagnosis device, and is characterized in that: the pickup image is separated into one or more noise component images and one or more signal component images; on at least one of the noise component images, frame synthesis processing is performed together with corresponding noise component images in one or more frames of an ultrasonic image of different time phases; and a noise synthetic image obtained through the frame synthesis image is synthesized with the signal component images.

Further, the present invention is characterized in that plural frames of an ultrasonic image including frames of at least two time phases are used to separate the ultrasonic image into a noise component image and a signal component image.

Further, the present invention is characterized in that parameters for frame synthesis are changed according to a region to be imaged or a scan rate.

The present invention is a method for improving image quality of a pickup image of an ultrasonic diagnosis device, and is characterized in that: the pickup image is separated into one or more noise component images and one or more signal component images; on at least one of the signal component images, frame synthesis processing is performed together with corresponding signal component images in one or more frames of an ultrasonic image of different time phases; on at least one of the noise component images, frame synthesis processing is performed together with corresponding noise component images in one or more frames of the ultrasonic image of different time phases; and a noise synthetic image and a signal synthetic image that are obtained through the pieces of frame synthesis processing are synthesized with each other.

Further, the present invention is characterized in that: magnitudes of positional deviations from a signal component image are calculated; after the magnitudes of positional deviations are compensated, frame synthesis processing is performed together with signal component images in one or more frames of an ultrasonic image of different time phases.

Further, the present invention is characterized in that: magnitudes of positional deviations from a signal component image calculated during the frame synthesis processing with signal component images are used to compensate magnitudes of positional deviations; and frame synthesis processing is performed together with noise component images in one or more frames of an ultrasonic image of different time phases.

Further, the present invention is characterized in that frame synthesis processing with signal component images and frame synthesis processing with noise component images are performed using different parameters.

Further, the present invention is characterized in that: two or more images that are different from each other in processing parameters are displayed; and the processing parameters are automatically set based on an image selected from among the displayed images or a region in the image.

### Advantageous Effects of Invention

According to an aspect of the present invention, in a method for improving image quality of an ultrasonic image and a device thereof, after an image is separated into a signal component image and a noise component image, frame synthesis processing is performed on the noise component image. Thus, both suppression of a noise and preservation of a signal component can be accomplished.

In addition, after an image is separated into a signal component image and a noise component image, frame synthesis processing is performed on the signal component image. Thus, discernment of a signal component can be improved.

In addition, an image is separated into plural signal component images and plural noise component images, and different pieces of processing are performed on the signal component images and noise component images respectively. Thus, appropriate processing can be performed according to a type of signal component or noise component.

In addition, parameters for frame synthesis processing are changed according to a scan rate or a region to be imaged. Thus, a high-quality image can be outputted under various conditions all the time.

In addition, two or more images that are different from one another in processing parameters are displayed. A user-selected image or information on a region in the image is used to automatically set the processing parameters. Thus, the processing parameters can be appropriately and readily set.

### BRIEF DESCRIPTION OF DRAWINGS

FIG. 1 is a diagram showing a sequence of image quality improvement processing in accordance with a first embodiment.

FIG. 2 is a diagram showing a flow of image quality improvement processing in accordance with the first embodiment.

FIG. 3 is a diagram showing a fundamental configuration of an ultrasonic diagnosis device in which image quality improvement processing in accordance with each of embodiments is implemented.

FIG. 4 is a diagram showing a sequence of image quality improvement processing in accordance with a third embodiment for a case where an image is separated into plural signal component images.

FIG. 5 is a diagram relating to a fourth embodiment and showing a sequence of image quality improvement processing for a case where an image is separated into plural noise component images.

FIG. 6 is a diagram relating to a fifth embodiment and showing a sequence of image quality improvement processing for a case where plural frames of an input image are used to perform signal/noise separation processing.

FIG. 7 is a diagram relating to a seventh embodiment and showing a sequence of signal/noise separation processing.

FIG. 8 is a diagram relating to an eighth embodiment and showing a sequence of image quality improvement processing for a case where frame synthesis processing is performed on a signal component image and a noise component image alike.

FIG. 9 is a diagram relating to a ninth embodiment and showing a sequence of image quality improvement processing for a case where positional deviation compensation processing and frame synthesis processing are performed on a signal component image.

FIG. 10 is a diagram relating to a tenth embodiment and showing a sequence of image quality improvement processing for a case where magnitudes of deviations calculated relative to a signal component image are used to compensate positional deviations of noise component images.

FIG. 11 is a diagram relating to an eleventh embodiment and showing a sequence of frame synthesis processing.

FIG. 12 is a diagram relating to a twelfth embodiment and explaining a weight calculation method employed in frame synthesis processing.

FIG. 13 is a diagram relating to a thirteenth embodiment and showing an adjustment screen image for use in adjusting parameters for image quality improvement processing.

FIG. 14 is a diagram relating to a fourteenth embodiment and showing an ultrasonic probe including an interface for parameter adjustment.

FIG. 15 is a diagram relating to a fifteenth embodiment and showing an adjustment screen image for use in adjusting parameters for image quality improvement processing.

FIG. 16 is a diagram relating to the fifteenth embodiment and showing a flow of adjusting parameters in the adjustment screen image shown in FIG. 15.

FIG. 17 is a diagram relating to a sixteenth embodiment and showing a setting screen image for use in automatically retrieving parameters for image quality improvement processing.

FIG. 18 is a diagram showing a flow of automatically retrieving parameters in a seventeenth embodiment.

FIG. 19 is a diagram relating to the eighth embodiment and showing a flow of image quality improvement processing for a case where frame synthesis processing is performed on a signal component image and a noise component image alike.

FIG. 20 is an embodiment diagram relating to the ninth embodiment and showing a flow of signal-component frame synthesis processing.

FIG. 21 is a diagram showing a sequence of image quality improvement processing in accordance with the second embodiment.

FIG. 22 is a diagram describing a flow of image quality improvement processing in accordance with the second embodiment.

FIG. 23 is a diagram showing a sequence of image quality improvement processing in accordance with the sixth embodiment for a case where frame synthesis processing is performed on a signal component image.

FIG. 24 is a diagram showing a flow of image quality improvement processing in accordance with the sixth embodiment for a case where frame synthesis processing is performed on a signal component image.

### DESCRIPTION OF EMBODIMENTS

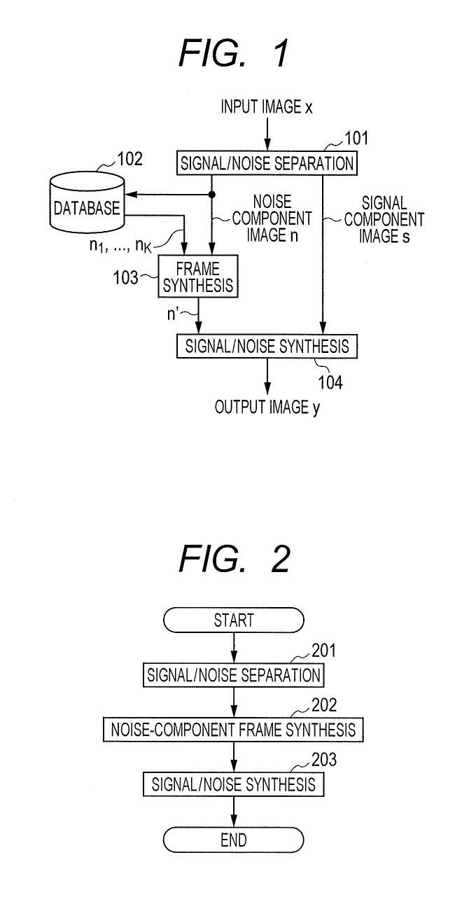

Various embodiments of the present invention will be described in conjunction with FIG. 1 to FIG. 24.

The present invention relates to processing and a device that perform image processing using an image of plural frames so as to improve image quality of a pickup image acquired by transmitting or receiving ultrasonic waves.

### First Embodiment

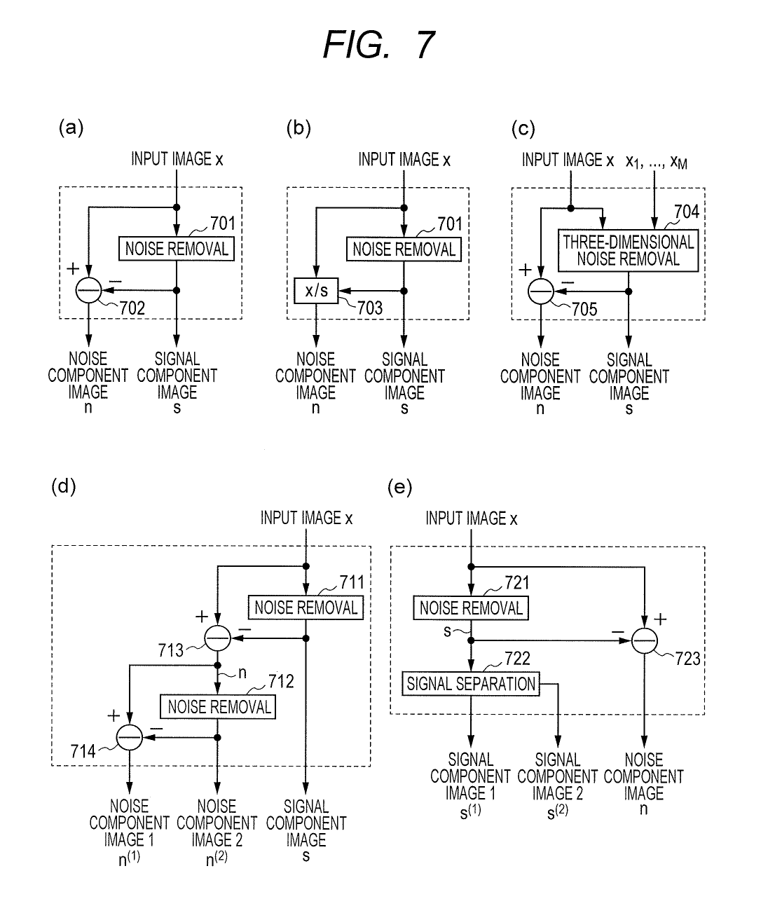

FIG. 1 is a diagram showing an example of a sequence of image quality improvement processing in accordance with a first embodiment. First, through signal/noise separation processing 101, an input image x is separated into a signal component image s and a noise component image n. A concrete example of the signal/noise separation processing 101 will be described later in conjunction with FIG. 7. The noise component image n is sequentially stored in a database 102. Thereafter, through frame synthesis processing 103, synthesis processing is performed on the noise component image n and K noise component images n₁, etc., and n_{K} of different time phases in order to obtain a noise synthetic image n′. The K noise component images of different time phases may be noise component images that precede the noise component image n by 1 to K time phases or may be noise component images whose time phases succeed the time phase of the noise component image n. Finally, through signal/noise synthesis processing 104, the signal component image s and noise synthetic image n′ are synthesized with each other in order to obtain an output image y. For example, in the signal/noise separation processing 101, separation is performed so that the sum of the signal component image s and noise component image n can be equal to the input image x. In the signal/noise synthesis processing 104, synthesis is performed so that the sum of the signal component image s and noise synthetic image n′ can be the output image y. However, the present invention is not limited to this mode.

Similarly to the present embodiment, when an input image is separated into a signal component image and a noise component image and frame synthesis processing is then performed on the noise component image, both suppression of a noise and preservation of a signal component can be achieved. Incidentally, noise removal processing capable of perfectly separating an input image x into a signal component and a noise component cannot be implemented. The noise component may be contained in a signal component image s, or a signal component may be in turn contained in a noise component image n.

Referring to the flowchart of FIG. 2, a description will be made of a flow of actions for image quality improvement processing in accordance with the present embodiment. First, through signal/noise separation processing 201, an input image is separated into a signal component image and a noise component image. Thereafter, through noise-component frame synthesis processing 202, frame synthesis processing is performed on the noise component image in order to obtain a noise synthesis image. Finally, through signal/noise synthesis processing 203, the signal component image is synthesized with the noise synthetic image in order to obtain an output image.

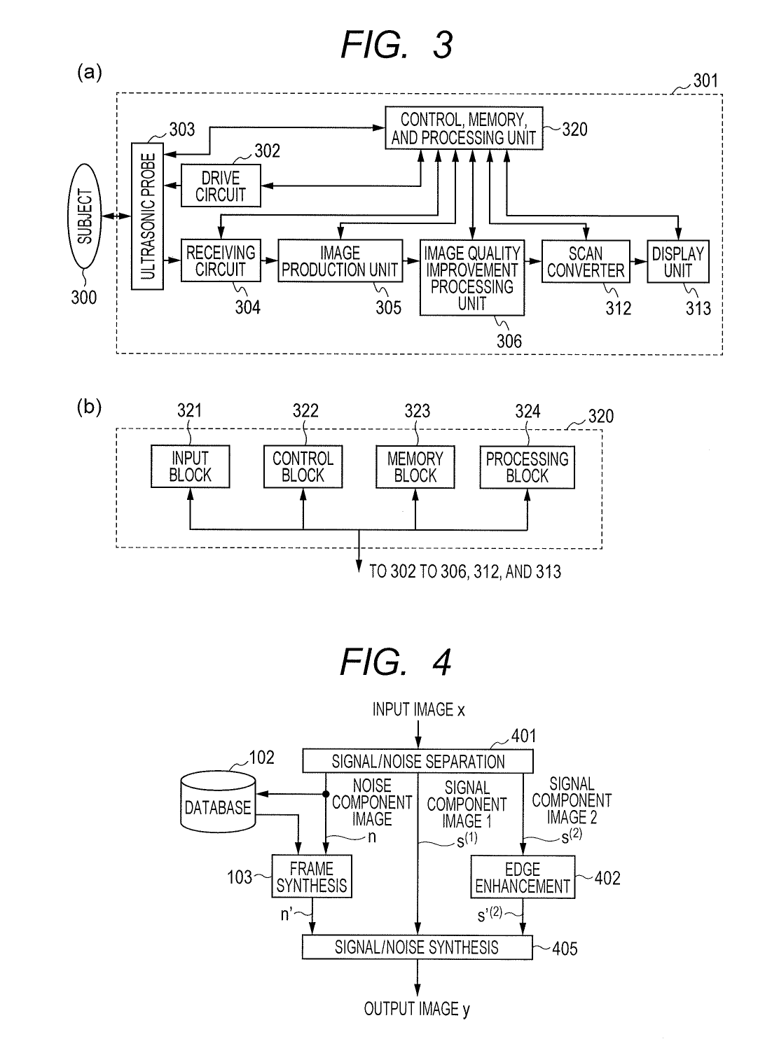

Next, an ultrasonic diagnosis device to which the present embodiment and other embodiments ace applied will be described in conjunction with FIG. 3. FIG. 3(a) is a diagram showing an example of a configuration of an ultrasonic diagnosis device 301. The ultrasonic diagnosis device 301 includes an ultrasonic probe 303 that transmits or receives ultrasonic signals, a drive circuit 302 that generates a driving signal to be inputted to the ultrasonic probe 303, a receiving circuit 304 that performs amplification of a receiving signal and analog-to-digital conversion, an image production unit 305 that produces an image having a scanning-line signal stream, which stems from ultrasonic scanning, arrayed two-dimensionally, an image quality improvement processing unit 306 that performs image quality improvement processing on an image, a scan converter 312 that performs coordinate conversion processing or interpolation processing on an image represented by the scanning-line signal stream, a display unit 313 that displays an image produced by the scan converter, and a control, memory, and processing unit 320 that controls all the components and stores and processes data. The ultrasonic probe 303 transmits an ultrasonic signal based on a driving signal to a subject 300, receives reflected waves that are obtained from the subject 300 during the transmission, and converts the reflected waves into an electric receiving signal. The ultrasonic probe 303 falls into types called, for example, linear, convex, sector, and radial types. When the ultrasonic probe 303 is of the convex type, the scan converter 312 transforms a rectangular image into a fan-shaped image.

The image quality improvement processing unit 306 is normally realized with, for example, a central processing unit (CPU), and can execute image quality improvement processing by running a program or the like. The sequence or flow of image quality improvement processing shown in FIG. 1 or FIG. 2 is implemented as software processing in the CPU or the like. Needless to say, the same applies to the sequence or flow of image quality improvement processing in each of embodiments to be described below.

The control, memory, and processing unit 320 includes, as shown in FIG. 3(b), as functional blocks an input block 321, a control block 322, a memory block 323, and a processing block 324. Normally, the control, memory, and processing unit 320 is realized with a CPU, a digital signal processor (DSP), or a field programmable gate array (FPGA). The input block 321 inputs parameters concerning the timing of beginning image production and production of an image. The control block 322 controls the actions of the drive circuit 302, ultrasonic probe 303, receiving circuit 304, and image quality improvement processing unit 306. In the memory block 323, a receiving signal, an image produced by the image production unit 305, an image calculated by the image quality improvement processing unit 306, and a display image that is an output of the scan converter 312 are stored. The processing block 324 performs reshaping processing on an electric signal to be inputted to the ultrasonic probe 303, and processing of adjusting a lightness and contrast for image display. In addition, the control, memory, and processing unit 320 may include the image quality improvement processing unit 306 as an internal facility thereof.

In the foregoing configuration, the ultrasonic probe 303 transmits an ultrasonic signal, which based on a driving signal controlled by the control block 322 of the control, memory, and processing unit 320, to the subject 300, receives a reflected signal obtained from the subject 300 due to the transmission, and converts the reflected signal into an electric receiving signal. The receiving signal converted into the electric signal is amplified and analog-to-digital converted by the receiving circuit 304. Thereafter, the analog-to-digital converted signal is processed by the image production unit 305 in order to produce an image, and the image is inputted to the image quality improvement processing unit 306. In the image quality improvement processing unit 306, signal/noise separation processing 101, frame synthesis processing 103, and signal/noise synthesis processing 104 are, as mentioned above, performed on the inputted image in order to thus carry out high-performance image quality improvement processing. An output image is then obtained. Further, the scan converter 312 performs image coordinate conversion processing or interpolation processing on the output image, and produces an image. Thus, a sharp ultrasonic image having a noise component thereof reduced can be displayed on the screen of the display unit 313. Incidentally, the present invention is not limited to the configuration of the present embodiment. For example, the image quality improvement processing unit 212 may be disposed on a stage preceding the scan converter 207.

### Second Embodiment

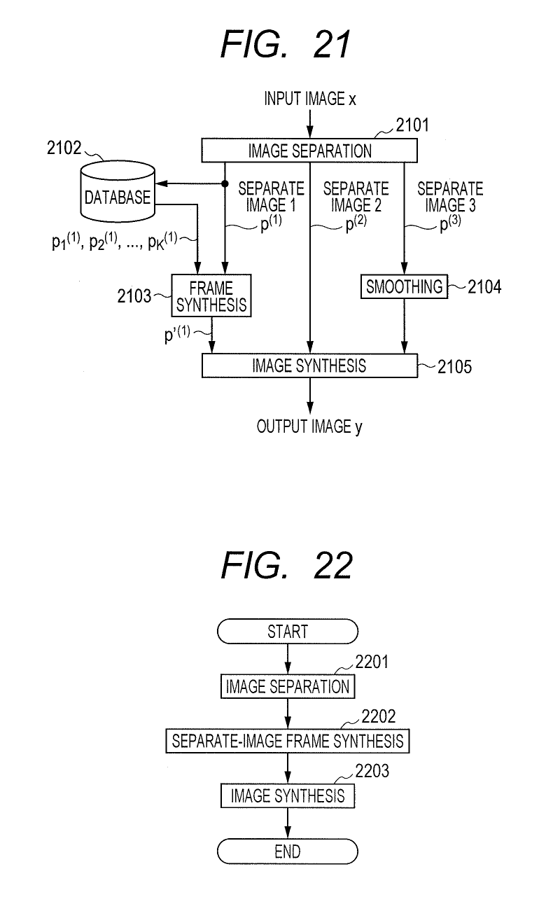

FIG. 21 is a diagram presenting a second embodiment and showing a sequence of image quality improvement processing different from the one in the first embodiment. First, through image separation processing 2101, an input image x is separated into three separate images 1 p⁽¹⁾, 2 p⁽²⁾, and 3 p⁽³⁾. Thereafter, the separate image 1 p⁽³⁾ that is one of the separate images is sequentially stored in a database 2102. Thereafter, through frame synthesis processing 2103, synthesis processing is performed on the separate image 1 p⁽¹⁾ and K separate images p₁⁽¹⁾, etc., and p_{K}⁽¹⁾ of different time phases in order to obtain a separate synthetic image p′⁽¹⁾. The K separate images 1 of different time phases may be separate images 1 that precede the separate image 1 p⁽¹⁾ by 1 to K time phases, or may include separate images 1 whose time phases succeed the time phase of the separate image 1 p⁽¹⁾. For example, smoothing processing 2104 to be performed on the separate image 3 p⁽³⁾ may be included. Finally, the separate synthetic image p′⁽¹⁾ and the other separate images are synthesized in order to obtain an output image y. For example, in the image separation processing, separation is performed so that the sum of the separate images 1 p⁽¹⁾, 2 p⁽²⁾, and 3 p⁽³⁾ can be equal to the input image x. In the image synthesis processing 2105, synthesis may be performed so that the sum of the separate images 1 p⁽¹⁾, 2 p⁽²⁾, and 3 p⁽³⁾ can be the output image y. However, the present invention is not limited to this mode. The present invention is not limited to the foregoing constitution. For example, the number of separate images to be produced through image separation processing may not be three, and plural separate images may be subjected to frame synthesis processing. Similarly to the present embodiment, an image may be separated into plural images, and frame synthesis processing may be performed on one or more separate images. Thus, while a component having a specific feature is suppressed and discernment of the component is improved, the remaining components can be preserved.

Referring to the flowchart of FIG. 22, a flow of actions for image quality improvement processing in accordance with the second embodiment will be described below. First, an input image is separated into plural separate images through image separation processing 2201. Thereafter, through separate-image frame synthesis processing 2202, separate-image frame synthesis processing is performed in order to obtain a separate synthetic image. Finally, through image synthesis processing 2203, the separate synthetic image is synthesized with the remaining separate images that do not undergo the frame synthesis processing in order to obtain an output image.

### Third Embodiment

FIG. 4 is a diagram relating to a third embodiment and showing a sequence of image quality improvement processing for a case where signal/noise separation processing is performed to separate an input image into plural signal component images and a noise component image. The same reference numerals as those in FIG. 1 are assigned to pieces of processing or data items which are identical to those in FIG. 1.

First, through signal/noise separation processing 401, an input image x is separated into two signal component images 1 s⁽¹⁾ and 2 s⁽²⁾ and a noise component image n. As for separation into plural signal component images, the separation can be performed based on a criterion, for example, a speed of a motion, a size of a structure, or a characteristic of a spatial frequency. As for the noise component image, similarly to the sequence in FIG. 1, through frame synthesis processing 103, synthesis processing is performed on the noise component image n and K noise component images n₁, etc., and n_{K} of different time phases in order to obtain a noise synthetic image n′. In addition, through edge enhancement processing 402, edge enhancement is performed on the signal component image 2 s(²) in order to obtain an image s′⁽²⁾. Thereafter, through signal/noise synthesis processing 405, the two signal component images s⁽¹⁾ and s⁽²⁾ and noise synthetic image n′ are synthesized with one another in order to obtain an output image y. In the present embodiment, edge enhancement procesing is performed on a signal component image on one side. Alternatively, any processing other than the edge enhancement processing may be performed, and processing may be performed on both the signal component images. Similarly to the present embodiment, when an input image is separated into plural signal component images, signal components can be preserved or enhanced with high performance. Thus, appropriate processing can be performed on the signal components.

### Fourth Embodiment

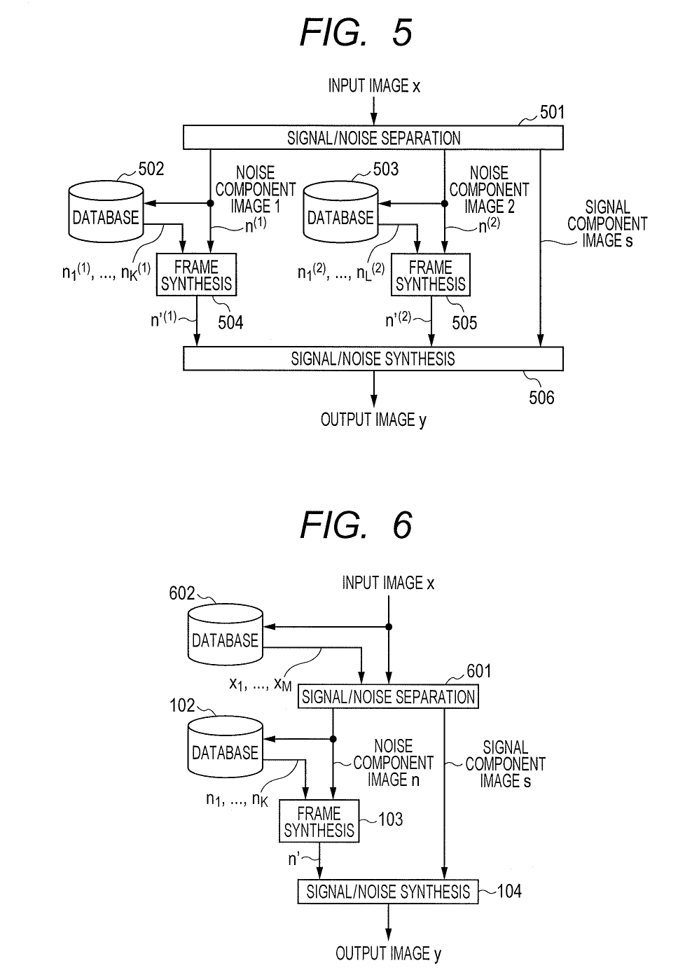

FIG. 5 is a diagram relating to a fourth embodiment and showing a sequence of image quality improvement processing for a case where signal/noise separation processing is performed to separate an input image into a signal component image and plural noise component images. First, through signal/noise separation processing 501, an input image x is separated into a signal component image s and two kinds of noise component images n⁽¹⁾ and n⁽²⁾. As for separation into plural noise component images, the separation can be performed based on a criterion, for example, a speed of a motion, a size of a structure, or a characteristic of a spatial frequency. As for the noise component image 1, through frame synthesis processing 504, synthesis processing is performed on the noise component image n⁽¹⁾ and K noise component images n₁⁽¹⁾, etc., and n_{K}⁽¹⁾ of different time phases in order to obtain a noise synthetic image n′⁽¹⁾. As for the noise component image 2, through frame synthesis processing 505, synthesis processing is performed on the noise component image n⁽²⁾ and L noise component images n₁⁽²⁾, etc., and n_{L}⁽²⁾ of different time phases in order to obtain a noise synthetic image n′⁽²⁾. Herein, K and L may denote different values. Between the frame synthesis processing 504 and frame synthesis processing 505, different methods may be used to perform synthesis or different sets of parameters may be used thereto. Thereafter, through signal/noise synthesis processing 506, the signal component image s is synthesized with the two kinds of noise synthetic images n′⁽¹⁾ and n′⁽²⁾ in order to obtain an output image y. Similarly to the present embodiment, when an input image is separated into plural noise component images, an effect of suppression of a noise component can be improved, that is, appropriate processing can be performed on the noise component.

Referring to FIG. 4 and FIG. 5, a description has been made of the sequences of separating an input image into two kinds of signal component images or two kinds of noise component images. Alternatively, an input image may be separated into three or more kinds of signal component images or three or more kinds of noise component images or separated into plural signal component images and plural noise component images.

### Fifth Embodiment

FIG. 6 is an embodiment diagram relating to a fifth embodiment and showing a sequence of image quality improvement processing for a case where plural frames of an input image are used to perform signal/noise separation processing. An input image x is sequentially stored in a database 602. Through signal/noise separation processing 601, the input image x and M input images x₁, etc., and x_{M} of different time phases are used to separate the input image x into a signal component image s and a noise component image n. The M input images of different time phases may be input images that precede by 1 to M time phases or input images whose time phases succeed the time phase of the input image x. Frame synthesis processing 103 and signal/noise synthesis processing can be performed in the same manner as those described in conjunction with FIG. 1. Similarly to the present invention, when plural frames of an input image are employed, the input image can be highly precisely separated into a signal component and a noise component.

### Sixth Embodiment

FIG. 7 is a diagram showing sequences of signal/noise separation processing in accordance with various exemplary embodiments which are implemented in the aforesaid embodiments. As for noise removal processing, an example is disclosed in Japanese Patent Application Laid-Open Publication No. 2008-278995 filed previously by the present inventor.

In FIG. 7(a), first, noise removal processing 701 is performed on an input image x in order to obtain a signal component image s. Thereafter, a noise component image n is obtained by performing processing 702 of subtracting the signal component image s from the input image x. Through the processing, separation can be achieved so that the sum of the signal component image s and noise component image n can be equal to the input image x. In FIG. 7(b), first, noise removal processing 701 is performed on an input image x in order to obtain a signal component image s. Thereafter, a noise component image n is obtained by performing processing 703 of dividing the input image x by the signal component image s. Through the processing, the product of the signal component image s by the noise component image n becomes equal to the input image x, and a noise can be regarded as a multiplicative noise. FIG. 7(c) is a diagram showing an embodiment for performing signal/noise separation processing using plural frames of an input image. First, an input image x and M input images x₁, etc., and x_{M} of different time phases are used to perform three-dimensional noise removal processing 704. After a signal component image s of the input image x is obtained, a noise component image n is obtained by performing processing 705 of subtracting the signal component image s from the input image x.

Further, FIG. 7(d) is a diagram showing an embodiment for separating an input image into a signal component image and plural kinds of noise component images. First, noise removal processing 711 is performed on an input image x in order to obtain a signal component image s. Thereafter, an image n is obtained by performing processing 713 of subtracting the signal component image s from the input image x. Thereafter, noise removal processing 712 is performed on the noise component image n in order to obtain a noise component image 2 n⁽²⁾. Thereafter, a noise component image 1 n⁽¹⁾ is obtained by performing processing 714 of subtracting the noise component image 2 n⁽²⁾ from the image n. Through the processing, separation can be achieved so that the sum of the signal component image s and two kinds of noise component images n⁽¹⁾ and n⁽²⁾ can be equal to the input image x. In the present embodiment, for example, in the noise removal processing 711 on the preceding stage, processing is performed under parameters under which noise removal is intensely performed. In the noise removal processing 712 on the succeeding stage, processing is performed under parameters under which noise removal is feebly performed. Thus, separation can be achieved so that the noise component image 1 n⁽¹⁾ can hardly contain a signal component, and the signal component image s can hardly contain a noise component.

FIG. 7(e) is a diagram showing an embodiment for separating an input image into plural kinds of signal component images and a noise component image. First, noise removal processing 721 is performed on an input image x in order to obtain an image s. Thereafter, a noise component image n is obtained by performing processing 723 of subtracting the image x from the input image x. Thereafter, signal separation processing 722 is performed on the image s in order to obtain two kinds of signal component images s⁽¹⁾ and s⁽²⁾. In the signal separation processing 722, separation can be achieved based on a criterion, for example, a speed of a motion, a size of a structure, or a characteristic of a spatial frequency.

### Seventh Embodiment

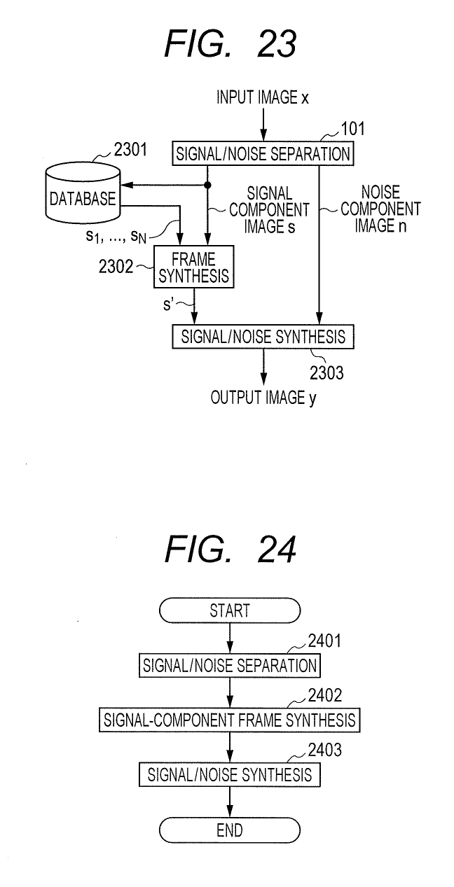

FIG. 23 is a diagram showing a sequence of image quality improvement processing in accordance with a seventh embodiment for a case where frame synthesis processing is performed on a signal component image. Signal/noise separation processing 101 is identical to the one in FIG. 1. A signal component image s is sequentially stored in a database 2301. Thereafter, through frame synthesis processing 2302, synthesis processing is performed on the signal component image s and N signal component images s₁, etc., and s_{N} of different time phases in order to obtain a signal synthetic image s′. The N signal component images of different time phases may be signal component images that precede the signal component image s by 1 to N time phases or may include signal component images whose time phases succeed the time phase of the signal component image s. Finally, through signal/noise synthesis processing 2303, the signal synthetic image s′ is synthesized with the noise component image n in order to obtain an output image y. Similarly to the present embedment, when an input image is separated into a signal component image and noise component image, and frame synthesis processing is performed on the signal component image, discernment of a signal component can be improved.

Referring to the flowchart of FIG. 24, a description will be made of a flow of actions for image quality improvement processing in accordance with the present embodiment for a case where frame synthesis processing is performed on a signal component image. First, an input image is separated into a signal component image and a noise component image through signal/noise separation processing 2401. Thereafter, through signal-component frame synthesis processing 2402, frame synthesis processing is performed on the signal component image in order to obtain a signal synthetic image. Finally, through signal/noise synthesis processing 2403, the signal synthetic image is synthesized with the noise component image in order to obtain an output image.

### Eighth Embodiment

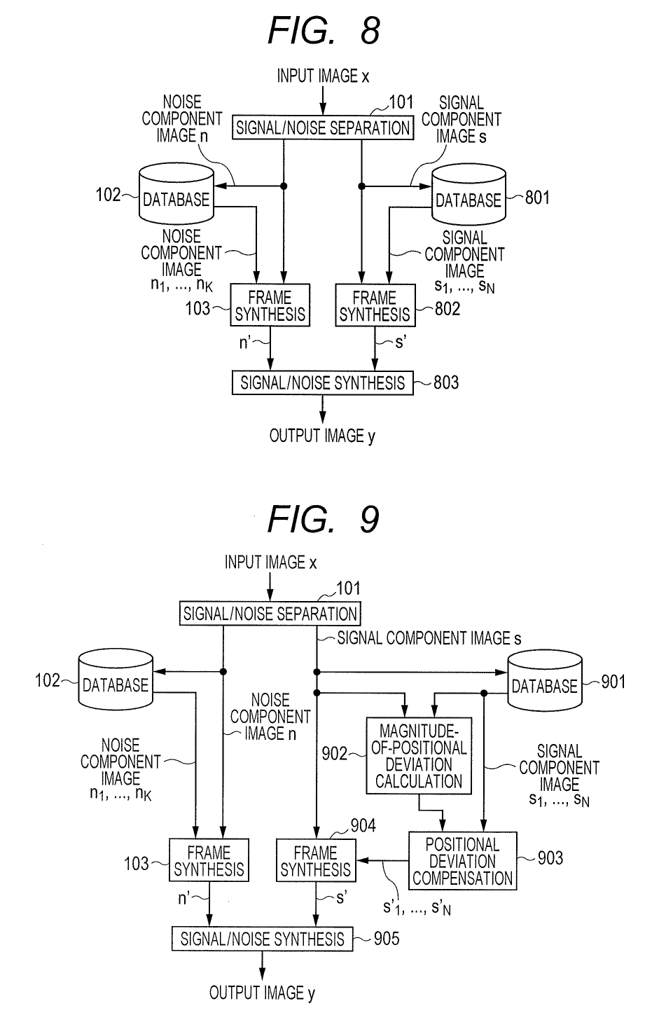

FIG. 8 is a diagram relating to an eighth embodiment and showing a sequence of image quality improvement processing for a case where frame synthesis processing is performed on a signal component image and a noise component image alike. Pieces of processing 101 to 103 are identical to those in FIG. 1. In the present embodiment, a signal component image s is also sequentially stored in a database 801. Through frame synthesis processing 802, synthesis processing is performed on the signal component image and N signal component images s¹, etc., and s^{N} of different time phases in order to obtain a signal synthetic image s′. Finally, through signal/noise synthesis processing 803, the signal synthetic image s′ is synthesized with a noise synthetic image n in order to obtain an output image y. The frame synthesis processing 103 and frame synthesis processing 802 may be performed according different methods or may be performed using different parameters for frame synthesis processing. Similarly to the present embodiment, when frame synthesis processing is performed on even a signal component image, discernment of a signal component can be improved.

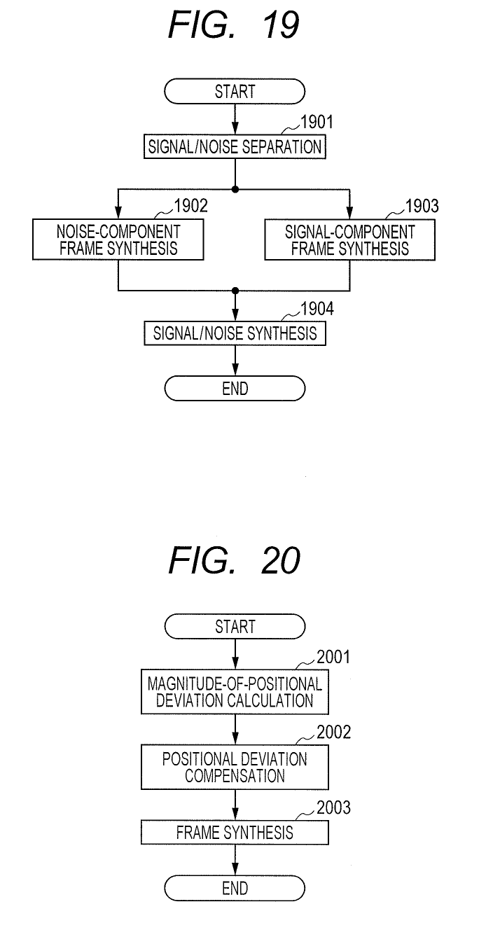

FIG. 19 is a diagram showing a flow of image quality improvement processing in accordance with the eighth embodiment, which is described in conjunction with FIG. 8, for a case where frame synthesis processing is performed on even a signal component image. First, an input image is separated into a signal component image and a noise component image through signal/noise separation processing 1901. Thereafter, through noise-component frame synthesis processing 1902, frame synthesis processing is performed on the noise component image in order to obtain a noise synthetic image. In addition, through signal-component frame synthesis processing 1903, frame synthesis processing is performed on the signal component image in order to obtain a signal synthetic image. Finally, through signal/noise synthesis processing 1904, the signal synthetic image is synthesized with the noise synthetic image in order to obtain an output image.

### Ninth Embodiment

FIG. 9 is a diagram relating to a ninth embodiment and showing a sequence of image quality improvement processing for a case where positional deviation compensation processing and frame synthesis processing are performed relative to or on a signal component image. The pieces of processing 101 to 103 are identical to those in FIG. 1. In the present embodiment, magnitudes of positional deviations of N signal component images s₁, etc., and s_{N} of different time phases from a signal component image s are calculated through magnitude-of-positional deviation calculation processing 902. For the calculation of the magnitudes of positional deviations, the magnitudes of deviations of the entire signal component images s₁, etc., and s_{N} may be obtained. Otherwise, an image may be divided into plural areas, and the magnitudes of deviations of the areas may be obtained. Thereafter, through positional deviation compensation processing 903, the calculated magnitudes of positional deviations are used to compensate the positional deviations of the signal component images s₁, etc., and s_{N} respectively. Thereafter, through frame synthesis processing 904, synthetic processing is performed on the signal component image s and the N signal component images s′₁, etc., and s′_{N}, which have the positional deviations thereof compensated, in order to obtain a signal synthetic image s′. Finally, through signal/noise synthesis processing 905, the signal synthetic image s′ is synthesized with a noise synthetic image n′ in order to obtain an output image y. Similarly to the present embodiment, when positional deviation compensation processing is performed relative to a signal component image, while blunting of a signal component is suppressed, discernment thereof can be improved. Similarly to the embodiment shown in FIG. 8, frame synthesis processing 904 may be performed according to a different method from the frame synthesis processing 103 is, or may be performed using different parameters for frame synthesis processing.

FIG. 20 is a diagram showing a flow of signal-component frame synthesis processing 904 in accordance with the present embodiment for a case where magnitudes of positional deviations of signal component images are compensated. First, magnitudes of positional deviations of N signal component images s₁, etc., and s_{N} of different time phases from a signal component image s are calculated through magnitude-of-positional deviation calculation processing 2001. Thereafter, through positional deviation compensation processing 2002, the calculated magnitudes of positional deviations are used to compensate the positional deviations of the signal component images s₁, etc., and s_{N} of different time phases. Finally, through frame synthesis processing 2003, synthesis processing is performed on the signal component image s and the N signal component images s′₁, etc., and s′_{N}, which have the positional deviations thereof compensated, in order to obtain a signal synthetic image s′.

### Tenth Embodiment

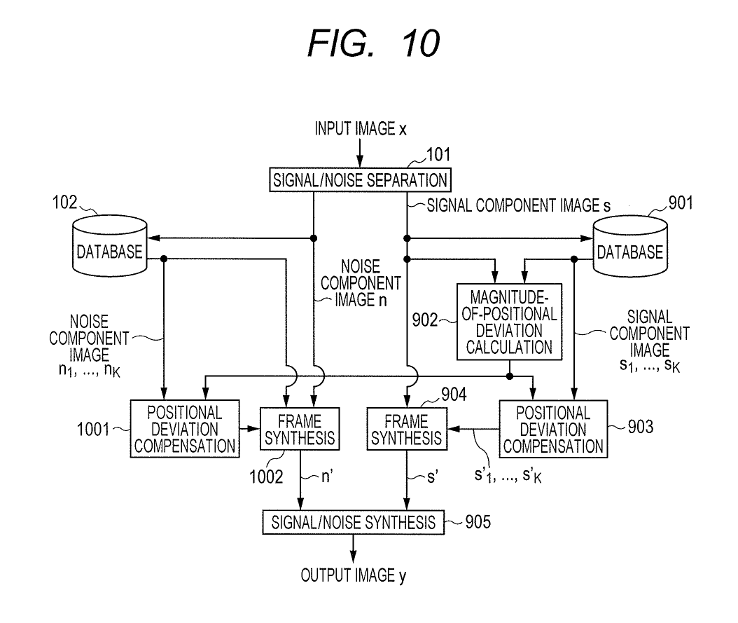

FIG. 10 is a diagram relating to a tenth embodiment and showing a sequence of image quality improvement processing for a case where magnitudes of deviations calculated relative to a signal component image are used to compensate positional deviations of noise component images. The pieces of processing 101, 102, and 901 to 905 are identical to those shown in FIG. 9. In the present embodiment, magnitudes of positional deviations calculated through magnitude-of-positional deviation calculation processing 902 are used to compensate positional deviations of noise component images n₁, etc., and n_{K} through positional deviation compensation processing 1001. Thereafter, through frame synthesis processing 1002, synthesis processing is performed on a noise component image n and the K noise component images n′₁, etc., and n′_{K}, which have the positional deviations thereof compensated, in order to obtain a noise synthetic image n′. In a noise component image, part of a signal component that has not been separated through signal/noise separation processing 101 may coexist. In this case, similarly to the present embedment, when positional deviation compensation processing is performed relative to a noise component image, while the signal component coexisting in the noise component image is preserved, a noise can be suppressed.

### Eleventh Embodiment

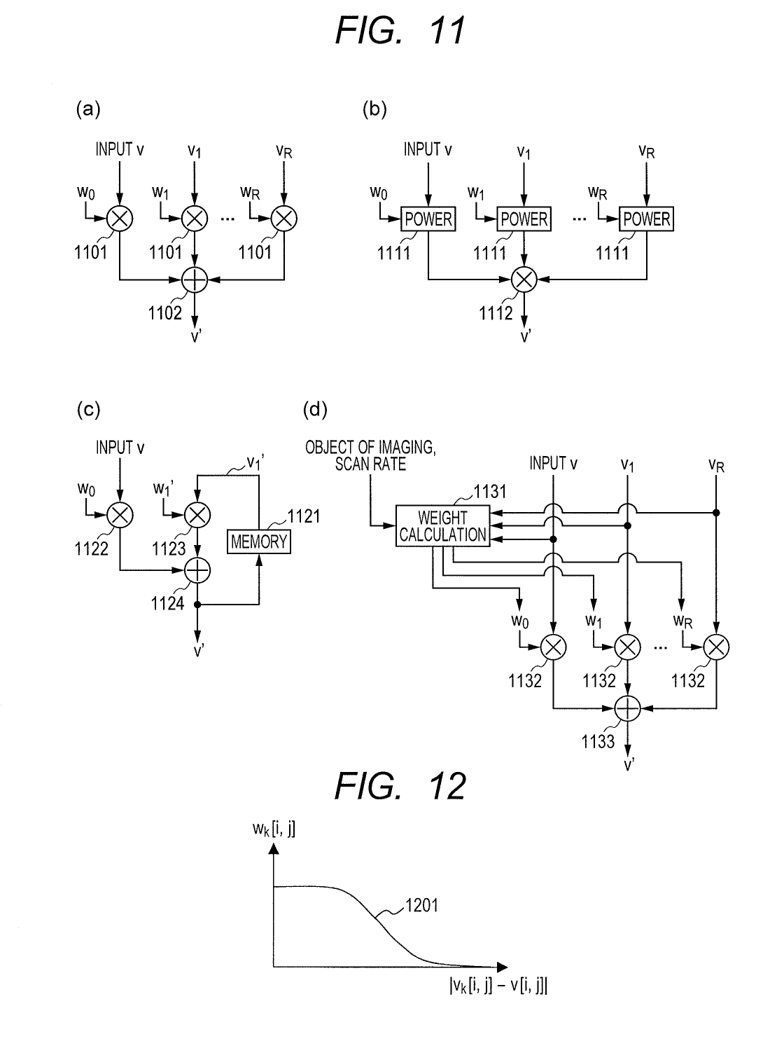

FIG. 11 is a diagram relating to an eleventh embodiment and showing various sequences of frame synthesis processing in the aforesaid embodiments. In FIG. 11(a), product-sum computation 1101 and 1102 is performed on an input v and R images v₁, etc., and v_{R}, time phases of which are different from the time phase of the input v, using weights w₀, etc., and w_{R} in order to obtain an image v′. In FIG. 11(b), as a calculation method other than the product-sum computation, after power computation 1111 is performed on the input v and images v₁, etc., and v_{R} using the weights w₀, etc., and w_{R}, multiplication 1112 is performed in order to obtain an image v′.

In FIG. 11(c), infinite response filter type computation is carried out. An image v′ is stored in a memory 1121. Product-sum computation 1122 to 1124 is performed on the image v1′, which is stored in the memory and previously obtained for one time phase, and an input, in order to obtain an image v′. In the present embodiment, only one frame of the image v′ is stored in the memory. Alternatively, plural frames of the image may be stored, and the image v′ and plural frames of the image of different time phases may be employed.

In FIG. 11(d), product-sum computation 1132 and 1133 is carried out in the same manner as that in FIG. 11(a). A difference lies in a point that weights w₀, etc., and w_{R} are modified through weight calculation processing 1131. In the weight calculation processing 1131, the weights are manually or automatically modified according to an object of imaging, that is, a region to be imaged, or a scan rate. Otherwise, the weights may be modified according to an input v and images v₁, etc., and v_{R}. When the weights are modified according to the object of imaging or scan rate, a high-quality image can always be outputted under various conditions. In frame synthesis processing, the weights w₀, etc., and w_{R} may take on values that differ from one pixel of an image to another, or may take on the same values for all pixels.

### Twelfth Embodiment

FIG. 12 is a diagram relating to a twelfth embodiment and showing a method of calculating weights w₀, etc., and w_{R} from an input v and images v₁, etc., and v_{R} during frame synthesis processing like the one shown in FIG. 11(d). In the present embodiment, a value wk[i,j] of a weight wk at a position [i,j] is obtained using a difference between the input v at the same position and an image vk thereat, |v[i,j]-vk[i,j]|. A graph 1201 indicates a relationship between the value Iv[i,j]-vk[i,j]| and weight wk[i,j]. In the present embodiment, the weights are designated so that the larger the difference between the value v[i,j] and value vk[i,j] is, the smaller the weight wk[i,j] is. In general, when a signal component is contained, the different tends to get larger. In this case, by decreasing the associated weight wk[i,j], blunting of the signal component can be prevented. In the present embodiment, the weight wk[i,j] monotonously decreases in relation to the value |v[i,j]−vk[i,j]|. However, the present invention is not limited to this relationship.

### Thirteenth Embodiment

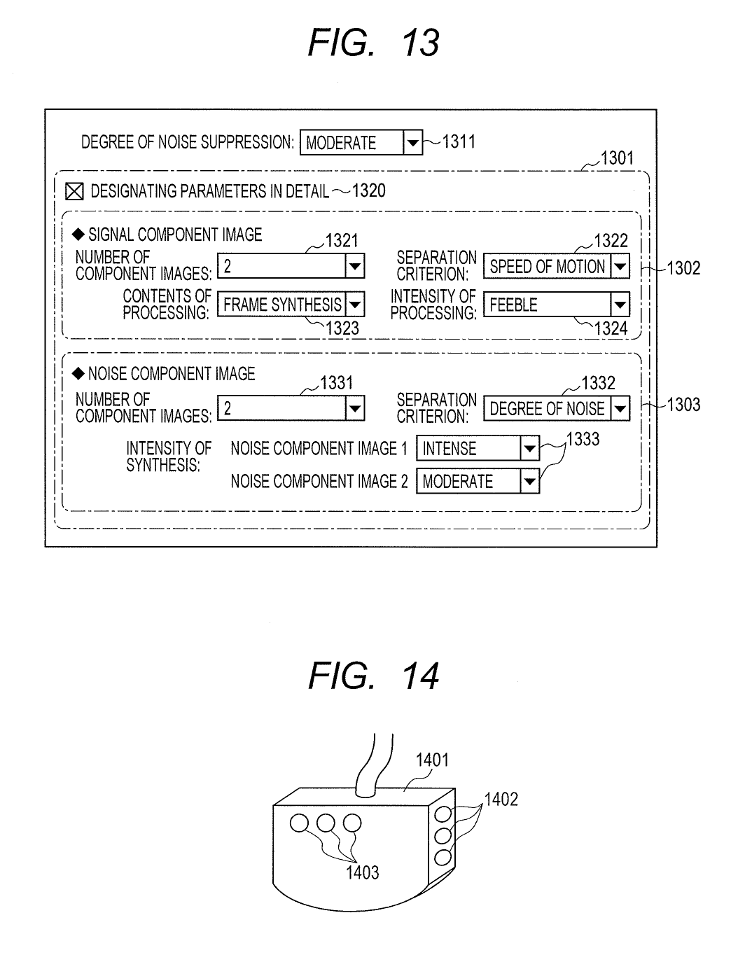

FIG. 13 is a diagram relating to a thirteenth embodiment and showing an adjustment screen image for use in adjusting parameters for image quality improvement processing. In the present embodiment, a field 1311 for designating a degree of noise suppression is included. Similarly to a field 1301, a field for designating parameters in detail may be included. Fields 1302 and 1303 are fields for adjusting parameters concerning processing of a signal component image and processing of a noise component image respectively. As for the processing of a signal component image to be designated in the field 1302, there are a field 1321 for the number of component images in which the number of signal component images into which an input image is separated is designated, a field 1322 for a separation criterion in which a criterion for separating the input image into plural signal component images is designated, a field 1323 for the contents of processing in which what processing should be performed after separation is designated, and a field 1324 for the intensity of processing in which the intensity of the contents of processing is designated. As for the processing of a noise component image to be designated in the field 1303, there are a field 1331 for the number of component images in which the number of noise component images into which an input image is separated is designated, a field 1332 for a separation criterion in which a criterion for separating the input image into plural noise component image is designated, and a field 1333 for the intensity of synthesis in which the intensity of frame synthesis processing is designated. In the present embodiment, an example of an interface for use in adjusting parameters is presented. Kinds of parameters capable of being designated, choices of values capable of being designated, a designation method, a layout, and others are not limited to those presented in the present embodiment.

### Fourteenth Embodiment

FIG. 14 is a diagram showing a fourteenth embodiment specific to an ultrasonic probe including an interface for parameter adjustment. In the present embodiment, buttons 1403 and buttons 1402 are disposed on the flanks of a probe 1401. Using the buttons, various kinds of parameters can be adjusted in, for example, the adjustment screen image shown in FIG. 13. The interface for parameter adjustment is not limited to the one of the present embodiment.

### Fifteenth Embodiment

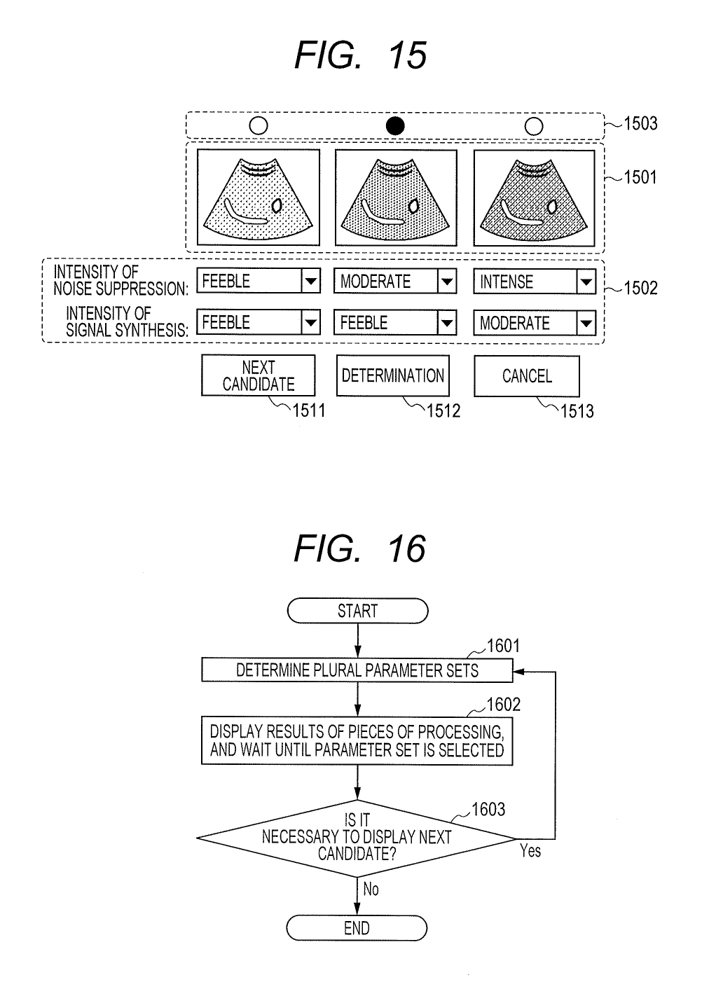

FIG. 15 is a diagram relating to a fifteenth embodiment and showing an adjustment screen image for use in adjusting parameters for image quality improvement processing. In the present embodiment, in a field 1501, the results of pieces of processing performed under three different kinds of sets of parameters are displayed simultaneously. The results of pieces of processing may be motion pictures or still images. The values of the parameters are displayed in a field 1502. Using a button 1503, one of the three kinds of parameters under which the best result is obtained can be selected. When a button 1511 is depressed, two kinds of parameters other than the selected parameters are changed to other parameters, and the results of pieces of processing are displayed.

FIG. 16 is a diagram presenting a flowchart of adjusting parameters in the adjustment screen image shown in FIG. 15. In the present flow, first, a block 1601 determines plural parameter sets (sets of parameters) serving as candidates. For the determination of the parameter sets, for example, a currently selected parameter set and parameter sets to be obtained by modifying part of the parameter set are obtained. Thereafter, a block 1602 displays the results of processing, and waits until a parameter set is selected. When a parameter set is selected, if a block 1603 decides that it is necessary to display the next candidates for parameter sets, processing of a block 1601 is carried out. The pieces of processing of the blocks 1601 to 1603 are repeated until it becomes unnecessary to display the next candidates for parameter sets. The case where it is unnecessary to display the next candidates for parameter sets refers to, for example, a case where a button other than the button 1511, which is used to display the next candidates, is selected in the adjustment screen image shown in FIG. 15, a case where there is no candidate that should be displayed next, or a case where a certain number of candidates or more have been displayed. Using the adjustment screen image shown in FIG. 15 or the flowchart of FIG. 16, a user can easily adjust the parameters.

### Sixteenth Embodiment

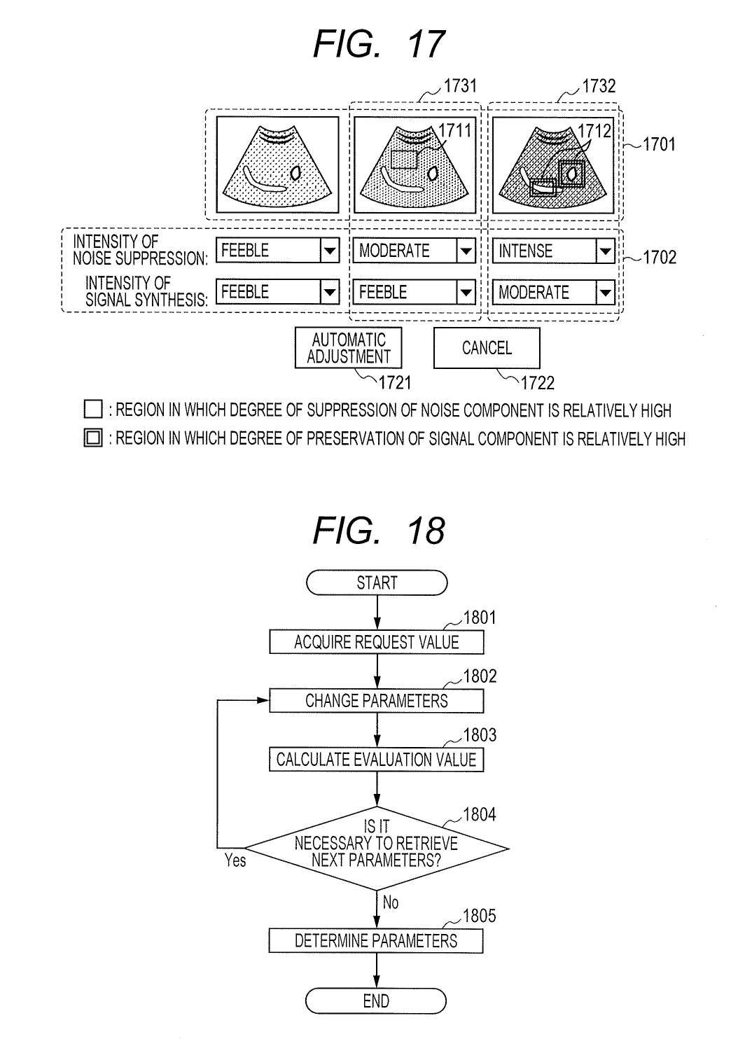

FIG. 17 is a diagram relating to a sixteenth embodiment and showing a setting screen image for use in automatically retrieving processing parameters for image quality improvement processing. In the present embodiment, in a field 1701, the results of pieces of processing performed under three different kinds of processing parameters are displayed. The results of pieces of processing may be motion pictures or still images. The values of the processing parameters are displayed in a field 1702. A selective region 1711 in an image is a region in which a degree of noise-component suppression is relatively high, a selective region 1712 is a region in which a degree of signal-component preservation is relatively high. An interface allowing a user to designate the intra-image selective regions 1711 and 1712 is included. As the intra-image selective region 1711 or 1712, a region in any of plural displayed images may be designated or plural regions may be designated.

When an automatic adjustment button 1721 is depressed in this state, parameters under which a degree of noise suppression is satisfactory in an intra-image selective region (hereinafter, a suppression-prioritized region) delineated as the region 1711 and a degree of signal enhancement is satisfactory in an intra-image selective region (hereinafter, a preservation-prioritized region) delineated as the region 1712 are automatically retrieved.

FIG. 18 is a diagram of a flowchart for automatically retrieving parameters in the sixteenth embodiment. First, a block 1801 acquires a request value that will be described later. Thereafter, a block 1802 changes current parameters. A block 1803 calculates an evaluation value for the changed parameters. When a degree of noise suppression in a suppression-prioritized region in a result of processing calculated using the current parameters is higher, the evaluation value is larger. When a degree of signal preservation in a preservation-prioritized region therein is higher, the evaluation value is larger. For example, the evaluation value can be calculated based on the sum of the degree of noise suppression in the suppression-prioritized region and the degree of signal preservation in the preservation-prioritized region. For example, the degree of noise suppression can be calculated to get larger as a quantity of a high-frequency component in an object region is smaller, and the degree of signal preservation can be calculated to get larger as the quantity of the high-frequency component in the object region is larger.

The request value refers to an evaluation value obtained based on the degree of noise suppression calculated from a region delineated as the region 1711 and the degree of signal preservation calculated from a region delineated as the region 1712 in the seventeenth embodiment. If a block 1804 decides that it is necessary to retrieve the next parameters, processing returns to processing of changing parameters to be performed by the block 1802. If it is unnecessary to retrieve the next parameters, parameter determination processing of a block 1805 is carried out, and parameter automatic retrieval processing is terminated. In the parameter determination processing, the current parameters are replaced with parameters for which the highest evaluation value is calculated in the course of retrieving parameters. As a criterion based on which the block 1804 decides whether it is necessary to retrieve the next parameters, a criterion such as whether the evaluation value is larger than the request value by a certain value or more or whether parameters are changed a certain number of times or more can be utilized. According to the present embodiment, in the adjustment screen image of FIG. 17, parameters under which both the degree of noise suppression like the one in the region delineated as the region 1711 and the degree of signal preservation like the one in the region delineated as the region 1712 become satisfactory can be retrieved in the adjustment screen image shown n FIG. 17.

Various embodiments of the present invention have been described so far. The present invention is not limited to the aforesaid embodiments but can be modified and implemented. For example, in the frame synthesis processing in FIG. 11, instead of synthesis through product-sum computation or power-product computation, synthesis may be performed using a more complex or simpler expression. In FIG. 15, instead of displaying the results of pieces of processing performed under three different kinds of parameters, the results of pieces of processing performed under two or four different kinds of parameters may be displayed.

### INDUSTRIAL APPLICABILITY

The present invention proves useful as an ultrasonic diagnostic device that acquires an image by transmitting or receiving ultrasonic waves to or from a subject, or more particularly, as a method for improving image quality, an ultrasonic diagnosis device, and a program for improving image quality which perform image quality improvement processing, that is image processing, on an acquired image.

### REFERENCE SIGNS LIST

101: signal/noise separation processing, 102: database, 103: frame synthesis processing, 104: signal/noise synthesis processing, 201: signal/noise separation processing, 202: noise-component frame synthesis processing, 203: signal/noise synthesis processing, 300: subject, 301: ultrasonic diagnosis device, 302: drive circuit, 303: ultrasonic probe, 304: receiving circuit, 305: image production unit, 306: image quality improvement processing unit, 312: scan converter, 313: display unit, 321: input block, 322: control block, 323: memory block, 324: processing block, 320: control, memory, and processing unit, 701: noise removal processing, 722: signal separation processing, 902: magnitude-of-positional deviation calculation processing, 903: positional deviation compensation processing, 1131: weight calculation processing.

1. A method for improving image quality of an ultrasonic image picked up by an ultrasonic diagnosis device, comprising:
separating a pickup image into two or more separate images;
performing frame synthesis processing on at least one of the separate images together with corresponding separate images in one or more frames of the pickup image of different time phases;
synthesizing a frame synthetic image, which is obtained by performing separate-image frame synthesis, with the separate image other than the separate image that has undergone the frame synthesis processing; and
displaying an image that stems from image synthesis.

2. The method for improving image quality of an ultrasonic image according to claim 1, wherein:
the pickup image is separated into one or more noise component images and one or more signal component images;
frame synthesis processing is performed on at least one of the noise component images together with corresponding noise component images in one or more frames of the pickup image of different time phases in order to obtain a noise synthetic image; and
the noise synthetic image obtained by performing noise-component frame synthesis is synthesized with the signal component image.

3. The method for improving image quality of an ultrasonic image according to claim 1, wherein:
processing parameters for frame synthesis processing including the number of frames to be synthesized or a weight are changed according to a region to be imaged or a scan rate set in the ultrasonic diagnosis device.

4. The method for improving image quality of an ultrasonic image according to claim 1, wherein:
the pickup image is separated into one or more noise component images and one or more signal component images;
frame synthesis processing is performed on at least one of the signal component images together with corresponding signal component images in one or more frames of the pickup image of different time phases in order to obtain a signal synthetic image; and
the signal synthetic image obtained by performing signal-component frame synthesis is synthesized with the noise component image.

5. The method for improving image quality of an ultrasonic image according to claim 4, wherein:
magnitudes of positional deviations of the corresponding signal component images in one of more frames of the pickup image of different time phases from the signal component image are calculated; and
after the magnitudes of positional deviations are compensated, frame synthesis processing is carried out.

6. The method for improving image quality of an ultrasonic image according to claim 4, further comprising:
performing frame synthesis processing on at least one of the noise component images together with corresponding noise component images in one or more frames of the pickup image of different time phases so as to obtain a noise synthetic image, wherein
the signal synthetic image obtained by performing signal-component frame synthesis is synthesized with the noise synthetic image obtained by performing noise-component frame synthesis.

7. The method for improving image quality of an ultrasonic image according to claim 1, wherein:
two or more images that are different from each other in processing parameters are displayed; and
the processing parameters are automatically set based on an image selected from among the plurality of displayed images or an intra-image selective region.

8. An ultrasonic diagnosis device using an ultrasonic image, comprising:
an ultrasonic probe that transmits or receives ultrasonic waves to or from a subject;
an image production unit that produces a pickup image;
an image quality improvement processing unit that improves the image quality of the pickup image; and
a display unit that displays an image which has undergone improvement processing performed by the image quality improvement processing unit, wherein
the image quality improvement processing unit separates the pickup image into two or more separate images, performs frame synthesis processing on at least one of the separate images together with corresponding separate images in one or more frames of the pickup image of different time phases, and synthesizes an obtained frame synthetic image with the separate image other than the separate image that has undergone the frame synthesis processing; and
the display unit displays an image stemming from synthesis performed by the image quality improvement processing unit.

9. The ultrasonic diagnosis device according to claim 8, wherein:
the image quality improvement processing unit separates the pickup image into one or more noise component images and one or more signal component images, performs frame synthesis processing on at least one of the noise component images, into which the pickup image is separated, together with corresponding noise component images in one or more frames of the pickup image of different time phases so as to obtain a noise synthetic image, and synthesizes the obtained noise synthetic image with the signal component image.

10. The ultrasonic diagnosis device according to claim 8, wherein:
the image quality improvement processing unit changes processing parameters for frame synthesis processing, which include the number of frames to be synthesized or a weight, according to a region to be imaged or a scan rate set in the ultrasonic diagnosis device.

11. The ultrasonic diagnosis device according to claim 8, wherein:
the image quality improvement processing unit separates the pickup image into one or more noise component images and one or more signal component images, performs frame synthesis processing on at least one of the signal component images together with corresponding signal component images in one or more frames of the pickup image of different time phases so as to obtain a signal synthetic image, and synthesizes the obtained signal synthetic image with the noise component image.

12. The ultrasonic diagnosis device according to claim 11, wherein:
the image quality improvement processing unit calculates magnitudes of positional deviations of the corresponding signal component images in one or more frames of the pickup image of different time phases from the signal component image, compensates the magnitudes of positional deviations, and then performs the frame synthesis processing.

13. The ultrasonic diagnosis device according to claim 11, wherein:
the image quality improvement processing unit performs frame synthesis processing on at least one of the noise component images together with corresponding noise component images in one or more frames of the pickup image of different time phases so as to obtain a noise synthetic image, and then synthesizes the obtained signal synthetic image with the noise synthetic image; and
the image quality improvement processing unit uses different processing parameters for frame synthesis processing between the signal-component frame synthesis and noise-component frame synthesis.

14. The ultrasonic diagnosis device according to claim 8, wherein:
the image quality improvement processing unit displays two or more images that are different from one another in processing parameters, and autonomously sets the processing parameters on the basis of an image selected from among the displayed images or an intra-image selective region.

15. A recording medium in which a program for improving image quality of a pickup image, which is run in an ultrasonic diagnosis device including an ultrasonic probe that transmits or receives ultrasonic waves to or from a subject, an image production unit that produces the pickup image, and a display unit that displays the pickup image, is recorded, wherein:
the program for improving image quality separates the pickup image into two or more separate images, performs frame synthesis processing on at least one of the separate images together with corresponding separate images in one or more frames of the pickup image of different time phases, and synthesizes an obtained frame synthetic image with the separate image other than the separate image that has undergone the frame synthesis processing; and
the display unit displays an image stemming from synthesis performed by the image quality improvement processing unit.
